(19) United States
(12) Patent Application Publication
(10) Pub. No.: US 2008/0202507 A1
(43) Pub. Date: Aug. 28, 2008

(54) Device for Generating a Jet of Liquid for Cleaning Wounds

(76) Inventor: Mark Brandli, Erlinsbach (CH)

Correspondence Address: BRIAN ROFFE, ESQ, 11 SUNRISE PLAZA, SUITE 303, VALLEY STREAM, NY, 11580-6111, US

(21) Appl. No.: 12/066,710

(22) PCT Filed: Sep. 14, 2006
(86) PCT No.: PCT/EP06/08938

§ 371 Date: Mar. 13, 2008

(30) Foreign Application Priority Data
Sep. 14, 2005 (CH) 01492/05

(51) Int. Cl. A61M 11/02; B65D 83/00; B65D 25/40

(52) U.S. Cl. 128/200.23; 222/399; 222/566

(57) ABSTRACT

A device (1) is used to generate a jet of liquid for pressurized irrigation or decontamination of wounds or infected areas of the body. It is provided with a liquid container (2), with means for generating a liquid pressure, and with a nozzle (5) which is arranged at the end of a flexible conduit (4) and from which the jet of liquid emerges. The liquid container (2) is designed as a pressurized container which can be connected via a valve (3) to a gas cartridge (11) or a compressed-air attachment and to which the flexible conduit (4) leading to the nozzle (5) can be joined. The liquid container (2) can be connected to the conduit (4) via a shut-off member or control member (7). A simple device (1) is thereby made available which can be easily transported and can also be sterilized without difficulty.

The invention relates to a device for generating a jet of liquid according to the preamble of claim 1.

The device is especially suitable for the pressurised irrigation and/or decontamination of surgical or chronic wounds and of infected and/or contaminated areas of the body.

A device of this type is disclosed, for example, in WO 00/59385. To generate a fluid pressure, a pumping set housed in a casing is provided, which is designed as a differential piston pump, the low-pressure part of which is connected to a pressure-supply means, e.g. a compressor, while the high pressure part is connected via a hose to a liquid container on the one hand and via a flexible conduit to a nozzle emitting the jet of liquid on the other.

The device according to WO 00/59385 is massive and heavy in design and not really suitable for outpatient treatments and/or mobile applications. It is also used for debridation, i.e. for the removal of tissue and fibrin- and necrotic layers and operated at considerably higher pressure. Hence correspondingly more expensive and more massive apparatus is necessary.

The present invention is based on the problem of creating a device of the aforementioned type which can easily be transported and is therefore suitable not only for in-patient use, but also for outpatient treatments and mobile applications.

This problem is solved according to the invention by a device with the features of claim 1.

Further preferred embodiments of the device according to the invention form the subject matter of the dependent claims.

Due to the fact that according to the invention the liquid container is designed as a pressurised container which can be connected via a valve to a gas cartridge or a compressed air outlet, to which the flexible conduit leading to the nozzle can be connected, while the liquid container can be connected to the conduit via a shut-off or regulator body, a simple, easily transportable and also easily sterilisable device is created which is also suitable for outpatient treatments and can be used, not only by doctors, but also by nursing staff, e.g. for home nursing (Spitex etc.). The device according to the invention is in particular especially suitable for the pressurised irrigation and decontamination of surgical and chronic wounds and for the irrigation of infected or contaminated areas of the body.

The invention is next explained in more detail with the aid of the drawing, which shows in purely schematic form:

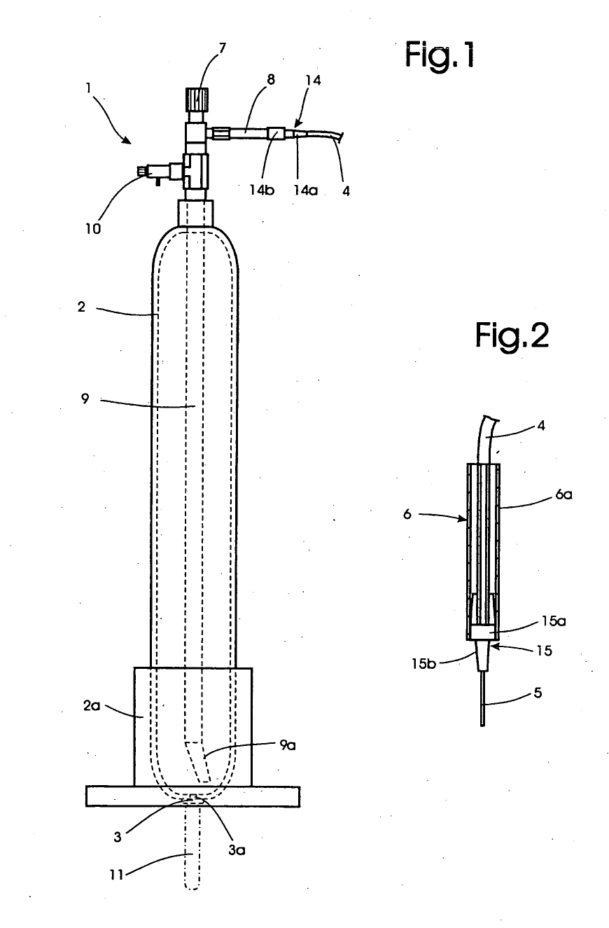

FIG. 1 an embodiment of a device according to the invention for generating a jet of liquid in lateral view; and

FIG. 2 a nozzle arranged on the end of a flexible conduit.

FIG. 1 shows a device 1 for generating a jet of liquid, for the pressurised irrigation and decontamination of surgical or chronic wounds or for pressurised irrigation of infected or contaminated areas of the body or similar.

The device 1 has a liquid container 2 with a stand 2a, which according to the invention is in the form of a, preferably bottle-shaped, pressurised container, and which can be connected via a valve 3 arranged in its base area to a compressed air source, in order to generate a liquid pressure. For mobile operation, the device 1 can use a gas cartridge 11 as compressed air source, while for in-patient operation compressed air can be fed in directly from a compressed air system outlet in the hospital.

The liquid container 2 contains a physiological solution or an antiseptic agent, for example a sterile saline solution or similar, which can be transported under pressure via a flexible conduit 4 attached to the liquid container 2 to a nozzle 5 fixed to the end of the conduit 4, said nozzle generating the jet of liquid. The nozzle 5, which can be seen from FIG. 2 and is described in more detail below, forms part of a hand piece 6 equipped with a handle 6a, which is provided for manual operation.

In the upper area of the device 1, above the liquid container 2, a shut-off or regulating body is arranged, preferably a regulating tap 7, with which the volume of liquid transported from the liquid container 2 via a connection piece 8 into the flexible conduit 4 can be adjusted. In the interior of the liquid container 2 is an immersion pipe 9, which is connected via the regulating tap 7 with the connection piece 8 and extends from the regulating tap 7 downwards, towards the container base, to above the gas fill opening 3a. The immersion pipe 9 is advantageously slightly angled at the lower end 9a, so that when filling with gas, as little gas as possible flows into the immersion pipe 9 and is thus wasted. The device 1 is also fitted with a ventilation means, preferably an adjustable breather valve (or vent tap) 10, by means of which air/gas can if necessary be suctioned out, so that the liquid container 2 can be filled more quickly.

The parts of the device 1 and/or of the liquid container 2 coming into contact with the liquid, including all fittings, are preferably made from stainless steel and can be sterilised both using steam and also in the plasma steriliser. The flexible conduit 4 can also be made of a suitable metal, although it is preferably made of plastic.

The flexible conduit 4 is preferably connected with a standard connection, e.g. using a so-called Luer-Lock connection 14 with the connection piece 8 connected downstream of this regulating tap 7, comprising a part fixed to the conduit 14a and also a part 14b assigned to the connection piece 8 (a union nut).

Also, the hand piece 6 arranged on the other end of the flexible conduit 4 and including the nozzle 5 is advantageously connected via a standard connection such as e.g. a Luer-Lock connection 15 to the conduit 4, in such a way that it cannot slip out under pressure. The conduit 4 is assigned a connection piece 15a (a union nut), the nozzle 5, which according to the invention is designed as a disposable needle or cannula made of plastic or metal, is connected with a connection piece 15b and can be disposed of with this after use. However, hand piece connections which do not correspond to a standard can easily be adapted, simply by loosening the union nut and attaching a suitable adapter.

The handle 6a already mentioned is attached to the connection piece 15a, preferably glued on, and this is tubular, for example made of plastic or cardboard.

The flexible conduit 4 and the connection pieces 14a, 15a assigned to it and e.g. cast with these and also the needle can—in accordance with FIG. 2—for example, be made of plastic.

The materials of the individual parts of the device 1 are selected such that they are resistant to heat, steam and all types of disinfectants, so that the metallic parts can be autoclaved using hot steam or can be disinfected using instrument disinfection agents. The disposable parts, which are preferably made of plastic, are plasma-sterilised or can be sterilised using gamma radiation.

The device according to the invention for generating a jet of liquid for the pressurised irrigation and decontamination of surgical or chronic wounds and of infected areas of the body or similar is simple and economical to manufacture and to assemble. It can be transported easily and hence is suitable not only for in-patient operation but also for outpatient treatments or mobile applications. It can be used not only by doctors, but also by nursing staff, e.g. for home nursing care (Spitex etc.).

1. Device to generate a jet of liquid for the pressurised irrigation or decontamination of wounds or infected areas of the body, the device is provided with a liquid container (2), with means for generating a liquid pressure, and with a nozzle (5) which is arranged at the end of a flexible conduit (4) and from which the jet of liquid emerges, characterised in that the liquid container (2) is designed as a pressurised container which can be connected via a valve (3) to a gas cartridge (11) or a compressed-air attachment and to which the flexible conduit (4) leading to the nozzle (5) can be joined, the liquid container (2) can be connected to the conduit (4) via a shut-off member or control member (7).

2. Device according to claim 1, characterised in that a drainage means (10) to suction out or allow gas/air to flow freely out of the liquid container (2) is provided, which is in the form of an adjustable drain valve or a drain tap.

3. Device according to claim 1, characterised in that the shut-off and/or control member (7) is in the form of a regulating tap.

4. Device according to claim 1, characterised in that an immersion pipe (9) is provided inside the liquid container (2), said immersion pipe being connected via the shut-off and/or control member (7) with a connection piece (8) for the flexible conduit (4).

5. Device according to claim 4, characterised in that the immersion pipe (9) of the shut-off and/or control member (7) extends downwards towards the base of the container, to above a gas fill opening (3a) and is preferably slightly angled at the lower end (9a).

6. Device according to claim 1 5, characterised in that the device is suitable for the pressurised irrigation and/or decontamination of surgical or chronic wounds and of infected and/or contaminated areas of the body.

7. Nozzle for a device according to claim 1, characterised in that the nozzle (5) has a disposable needle or cannula with a needle-shaped design, which is arranged on the end of the flexible conduit (4) by means of connector (15).

8. Nozzle according to claim 7, characterised in that a tubular handle (6a) is positioned on a connection piece (15a) of the connector (15) which is permanently attached to the end of the flexible conduit (4) and is permanently attached hereto.

9. Nozzle according to claim 7, characterised in that the disposable needle or cannula with a needle-shaped design is made of plastic or metal.
